(19) United States
(12) Patent Application Publication
(10) Pub. No.: US 2004/0010217 A1
(43) Pub. Date: Jan. 15, 2004

(54) Friction enhancing apparatus and method of application thereof

(76) Inventors: Russell E. Blette, Hastings, MN (US); E. Dale Good, White Bear Lake, MN (US); Patrick F. McClernon, White Bear Lake, MN (US)

Correspondence Address: 3M INNOVATIVE PROPERTIES COMPANY, PO BOX 33427, ST. PAUL, MN, 55133-3427, US

(21) Appl. No.: 10/196,562

(22) Filed: Jul. 15, 2002

(51) Int. Cl. A61F 13/00

(52) U.S. Cl. 602/54

(57) ABSTRACT

An apparatus for presenting an area of desired frictional interface on a portion of a user's appendage (e.g., hand or fingers) includes a flexible layer of material having first and opposite sides, with the first side having a frictional engagement surface. The apparatus further includes a flexible backing sheet having first and second opposite faces. A pressure sensitive adhesive is disposed on the second face of the backing sheet. The backing sheet has a central section and opposed tab portions, with the second side of the layer of material affixed to the first face of the backing sheet, in at least a portion of the central section thereof. The tab portions are sufficiently long, in dimension extending away from the central section, so that when the second face of the backing sheet is adhered to a user's appendage, the tab portions extend at least half way around the appendage. The frictional engagement surface has a static coefficient of friction of at least 1.1. The apparatus has a load ratio between a shear force design load an an anticipated load of at least 1.3:1.

### BACKGROUND OF THE INVENTION

The present invention relates to a patch of friction enhancing material that is removeably attached onto a user (such as on a finger), and to a method for accomplishing such attachment.

Many activities require the manipulating of an article with some degree of confidence, certainty and dexterity. Such activities may be work-related (e.g., handling materials such as boxes, cartons, tools, paper sheets, rolls of material, or a manual engaging surface such as a handle or railing) or may be sports-related (e.g., handling a ball, racquet handle, bat, or other sports grip or surface such as a fishing rod handle, ski handle, etc.). In many of these applications, an enhanced frictional interface between a user's hand and the article being manipulated or touched is desired, providing a more aggressive frictional interface than that available from mere direct contact between the user's skin and the article.

Various friction enhancement articles have been proposed for enhancing manual gripping characteristics. This includes gloves, or the application of a friction enhancing material to the user's hand (e.g., “stick-em”), or finger cots or stalls (coverings for fingertips). These approaches tend to cover most, if not all, of the useful tactile portions of the user's fingers, and are often uncomfortable in use as well. It has been proposed that small patches of friction-enhancing material be adhesively adhered adjacent the tips of the user's fingers for enhancing the user's ability to manipulate papers. Examples of such devices are seen in the following references: Yonkers U.S. Pat. No. 3,985,383, Scott U.S. Pat. No. 3,283,888, and Powell U.S. Pat. No. 5,547,465. The stated purpose of each of these devices is to increase the frictional interface between the user's finger and the articles being handled (e.g., paper). However, if the adhesive holding the finger patch is not tacky enough, the device can become dislodged from the finger in use. Thus, for such a patch the adhesive must not be so tacky as to make the article unremovable from the user's finger, but yet must be tacky enough to withstand the opposing frictional interface between the patch and the article being handled. Consequently, the ability to provide a high coefficient of friction interface between the patch and the article being handled is limited.

### BRIEF SUMMARY OF THE INVENTION

In one embodiment, the invention is an apparatus for presenting an area of desired frictional interface on a portion of a user's appendage which comprises a flexible layer of material and a flexible backing sheet. The flexible layer of material has first and second opposite sides, with the first side having a frictional engagement surface. The flexible backing sheet has first and second opposite faces, a central section and opposed tab portions. The second side of the flexible layer of material is affixed to the first face of the backing sheet, in at least a portion of the central section thereof. The apparatus also includes a pressure sensitive adhesive disposed on the second face of the backing sheet, wherein the tab portions of the backing sheet are sufficiently long, in dimension extending away from the central section, so that when the second face of the backing sheet is adhered to a user's appendage, the tab portions overlap at the free ends thereof.

In another embodiment, the present invention is an apparatus for presenting an area of frictional interface on a portion of the user's appendage which comprises a flexible layer of material and a flexible backing sheet. The flexible layer of material has first and second opposite sides, with the first side having a frictional engagement surface. The backing sheet has first and second opposite faces, a central section and opposed tab portions. The second side of the flexible layer of material is affixed to the first face of the backing sheet, in the central section thereof. The tab portions of the backing sheet are sufficiently long, in dimension extending away from the central section, so that when the second face of the backing sheet is adhered to a user's appendage, the tab portions extend at least half way around the appendage.

In another embodiment, the present invention is an apparatus for disposing a desired frictional interface on a user's appendage which comprises a flexible backing sheet having first and second opposite faces, with the backing sheet having a central section and opposed tab portions. The apparatus also comprises a pressure sensitive adhesive disposed on the second face of the backing sheet, wherein the tab portions of the backing sheet are sufficiently long, in dimension extending away from the central section, so that when the second face is adhered to a user's appendage, the tab portions extend at least half way around the appendage. The apparatus further comprises a friction zone defined on the first face of the backing sheet, with the friction zone having a static coefficient of friction of at least 1.1.

In another embodiment, the present invention is an apparatus for disposing a desired frictional interface on a portion of a user's appendage which comprises a flexible backing sheet having first and second opposite faces, means for removably attaching the second face of the flexible backing sheet to a user's appendage, and a friction zone defined on the first face of the backing sheet, wherein the friction zone has a static coefficient of friction of at least 1.1, and wherein the apparatus has a load ratio between a shear force design load and an anticipated load of at least 1.3:1.

In another embodiment, the present invention is a method for presenting a desired frictional interface on a user's appendage, comprising the steps of: (1) providing a flexible backing sheet having first and second opposite faces, the backing sheet having a central section and a plurality of tab portions extending outwardly from the central section, with the central section having, on its first face, a friction zone having a static coefficient of friction of at least 1.1, (2) exposing the pressure sensitive adhesive on the second face of the backing sheet, (3) aligning the backing sheet relative to a user's appendage for application, and (4) pressing the exposed pressure sensitive adhesive against the user's appendage to adhere the backing sheet thereto.

In another embodiment, the present invention is a method for presenting a desired frictional interface between a user's appendage and an article, comprising the steps of: (1) securing a bottom face of a flexible backing sheet to a user's appendage, wherein the backing sheet has, on a top face opposite the bottom face, a friction zone having a static coefficient of friction of at least 1.1, and (2) contacting an article with the friction zone for manipulation of the article.

### BRIEF DESCRIPTION OF THE DRAWINGS

The present invention will be further explained with reference to the drawing figures listed below, wherein like structures are referred to by like numerals throughout the several views.

FIG. 1 is a plan view of a first embodiment of a fingertip gripping pad of the present invention.

FIG. 2 is a sectional view as taken along lines 2-2 in FIG. 1.

FIG. 3A is an enlarged sectional view of one embodiment of the frictional engagement surface of the present invention.

FIG. 3B is an enlarged sectional view of the friction engagement surface of FIG. 3A in shear engagement with the surface of an article.

FIG. 4 is an enlarged photo illustration of one embodiment of the friction engagement surface of the present invention in frictional and shear engagement with the surface of an article (in this illustration, the article has a knurled metal surface).

FIGS. 5A-5D illustrate one form of the inventive method for applying a fingertip gripping pad of the present invention to a user's finger.

FIG. 6 is a side elevational view of a user's finger with the fingertip gripping pad of the first embodiment mounted thereon.

FIG. 7 is a sectional view as taken along lines 7-7 in FIG. 6 (with the adhesive layers not shown, for clarity).

FIG. 8 is a perspective view of an alternative embodiment of the fingertip gripping pad of the present invention, showing a plurality of such pads disposed on a common liner which is wound for storage and dispensing in roll form.

FIG. 9 is a plan view of another alternative embodiment of the fingertip gripping pad of the present invention.

FIG. 10 is a sectional view as taken along lines 10-10 in FIG. 9.

FIG. 11 is a schematic illustration of a Z-fold dispensing arrangement for the fingertip gripping pad of FIG. 9.

FIG. 12 is a plan view of a separable strip dispensing arrangement for the fingertip gripping pad of FIG. 9.

While the above-identified drawing figures set forth several embodiments of the invention, other embodiments are also contemplated, as noted in the discussion. In all cases, this disclosure presents the present invention by way of representation and not limitation. It should be understood that numerous other modifications and embodiments can be devised by those skilled in the art which falls in the spirit and scope of the principles of this invention.

### DETAILED DESCRIPTION

A primary objective of the present invention is to provide an apparatus and method of its application for enhancing the frictional interface between a portion of the user's appendage (i.e., a finger) and an article being manipulated by the user. The invention has particular utility in connection with manual activities where tactile acuity is desired, but there is a need for enhanding the frictional interface between the user's hand and the article being grasped. In addition, many such activities are conducted in conditions where fluids may be present, so an enhanced frictional interface is desired in both wet and dry conditions. For example, in many surgical or medical applications, it is important for the user (i.e., doctor or nurse) to be able to firmly grip an article yet still have high tactile sensation, in spite of the fact that various fluids may be present in the operating arena. Likewise, in many sports applications, it is important for the participant to firmly grip an article (such as a basketball or racquet handle) in spite of high and sudden forces being applied, and in wet and dry conditions (either wet from perspiration or from environmental conditions).

The apparatus of the present invention is quite useful for attaining these objectives. In the form of a fingertip gripping pad, the inventive apparatus is small, lightweight and flexible. It does not cover the entire hand or even an entire finger (like a typical finger cot or glove), thereby allowing some finger exposure for tactile feel of a tool or ball. The inventive apparatus conforms to the shape of the finger surface, and is thin so it does not unnecessarily “pad” the finger, which would diminish tactile sensation.

The inventive apparatus allows the presentation of a high frictional interface on a desired portion of a user's hands (i.e., a high coefficient of friction at the hand/article interface). The inventive apparatus achieves this objective without significantly comprising the user's sense of touch, and while still allowing for ease of application and removeability of the article from the user's fingers. In use, even in a very hostile environment (e.g., a basketball game where constant motion and momentary high sheer and normal forces are applied at the frictional interface), the fingertip gripping pad of the present invention stays in its desired location on the user's finger. In spite of the high and sudden forces applied against the fingertip gripping pad, it does not migrate or become dislodged in use. Among the advantages achieved by the present invention are that it provides an apparatus representing an area of desired frictional interface on a portion of the user's appendage which is relatively simple and inexpensive to manufacture, easy to apply and manipulate, and rugged and durable in use.

While the disclosure herein is primarily in the context of a fingertip gripping pad, the inventive apparatus is applicable to any user appendage, such as hand, wrist, forearm, elbow, bicep, shoulder, neck, head, thigh, knee, shin, ankle, foot or toes. The inventive apparatus is useful wherever spot or local temporary enhancement of the frictional interface of a user's appendage is desired, for whatever purpose. The inventive apparatus is easily applicable to an appendage, and is readily removable as well. For instance, applying a gripping pad to a portion of a user's foot provides an enhanced frictional interface on the foot for use in surfing. In addition, the inventive apparatus can be applied to a user's bare skin, or to other items already worn or otherwise covering the user's skin on an appendage (e.g., as a shoulder pad for a jacket, shirt or vest engaging a rifle butt in a shooting activity, or as an addition to a glove such as rubber surgical glove). As a further example, the inventive apparatus can be applied to bare fingers or a hand, or on portions of a glove, for use in a material handling application (e.g., handling piece parts in an industrial setting or handling shipping packages).

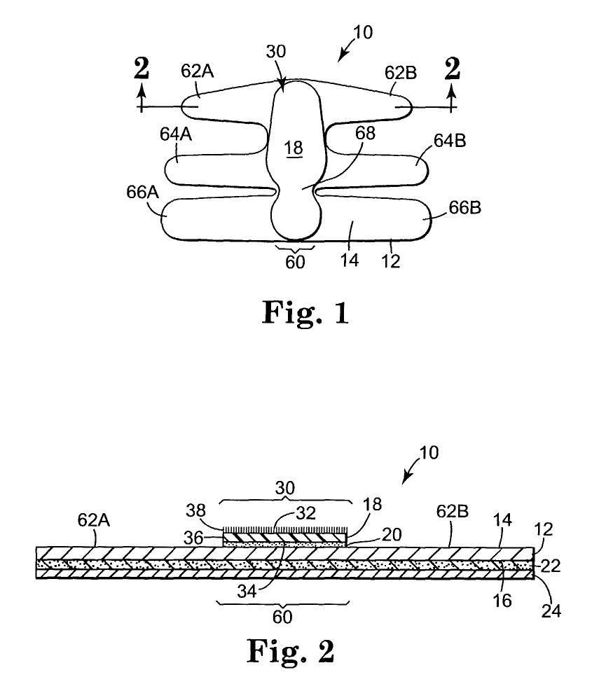

The drawing figures of this application are provided for illustrative purposes to disclose the nature and characteristics of the present invention. FIGS. 1 and 2 illustrate a first embodiment of the inventive friction enhancing apparatus, in the form of a fingertip gripping pad. In the embodiment illustrated in FIGS. 1 and 2, a gripping pad 10 has a backing sheet 12 which has a first side 14 and a second side 16. A layer of frictional material 18 is affixed on the first side 14 of the backing sheet 12 by adhesive 20. A second side 16 of the backing sheet 12 has adhesive 22 thereon which, prior to application to a user's finger, has an adhesive liner 24 adhered thereto.

The backing sheet 12 functions to support the layer of frictional material 18. The backing sheet 12 is preferably relatively thin, has a generally uniform thickness and is flexible, thereby allowing it to conform to the user's surface to which it is being adhered. The backing sheet must be relatively strong and durable in order to hold up under the possibly rigorous conditions of use. In one embodiment, the backing sheet is stretchable to further allow it to conform during application and use to the user. Preferably, the backing sheet is a comfortable material for adhering to a user's skin, being a breathable or permeable or perforated material. In medical or other applications (i.e., food handling), the backing sheet is sterilizable. Possible materials for use as the backing sheet 12 of the present invention include, but are not limited to, film, foam, woven, nonwoven, or a melt blown material. Specific materials contemplated for this purpose include the backings on the following products, all available from 3M Company, St. Paul, Minn., Transpore®, Comfort™, Medipore®, Micropore®, Active®, Tegaderm®, and Blenderm®.

The adhesive 22 on the second side 16 of the backing sheet 12 serves to adhere the fingertip gripping pad 10 to the user, which in some embodiments means directly to the user's skin. The adhesive 22 is preferably a pressure sensitive adhesive which is readily removable from the user's skin without adverse consequences or residue. The desired adhesive is tacky in both wet and dry conditions, and preferably is provided in a thin, generally uniform layer which covers all or part of the second side 16 of the backing sheet 12. In addition, the adhesive 22 is flexible which allows it to conform to the irregularities of a user's finger in use, and may be hypoallergenic and/or sterilizable. Possible adhesives include, but are not limited to, acrylate pressure sensitive adhesives (with or without tackifier), tackified styrene-diene pressure sensitive adhesives, polyolefin pressure sensitive adhesives (with or without tackifier), and silicon pressure sensitive adhesives.

The adhesive liner 24 is provided to cover all exposed adhesive 22 on the second side 16 of the backing sheet 12 prior to application of the fingertip gripping pad 10 to a user's finger. The adhesive liner 24 is flexible and is readily removeable from the adhesive 22, and may also be sterilizable. The side of the adhesive liner which contacts the adhesive 22 is provided with an adhesive release coat (such as silicone) to facilitate its separation from the adhesive 22. Possible materials for the adhesive liner 24 include, but are not limited to, paper, film, woven, or nonwoven.

The layer of frictional material 18 defines a friction zone 30 on the first side 14 of the backing sheet 12. The friction zone 30 is shaped to present the desired area of frictional interface between the user and the article being manipulated. As seen in FIG. 2, the layer of frictional material 18 has a first side 32 which is exposed and presents a desired frictional surface and a second side 34 which is contacted by adhesive 20 for adhering the layer of frictional material 18 to the backing sheet 12. The friction zone 30 thus provides the requisite frictional surface to achieve the desired frictional interface characteristics of the present invention in use.

In one embodiment, the layer of frictional material 18 is a base layer 36 which on its exposed working surface has an array of upstanding stems 38 projecting therefrom. Materials of this type are formed by the techniques disclosed in U.S. Pat. No. 6,372,323, and Applicant's co-pending U.S. patent application Ser. No. 09/637,567. Each stem in the array is flexible and durable. Upon the application of shear forces, the stems bend over, which actually enhances their frictional interface surface area against an opposed article. In the illustrated embodiment in FIGS. 1 and 2, the layer of frictional material 18 and the backing sheet 12 are bonded together by adhesive 20. A suitable adhesive for this purpose is Scotch® type 300 LSE high-strength adhesive, available from 3M Company, St. Paul, Minn. In one embodiment, however, the layer of frictional material 18 (and thus the array of upstanding stems 38 thereon) is integrally formed with the backing sheet 12.

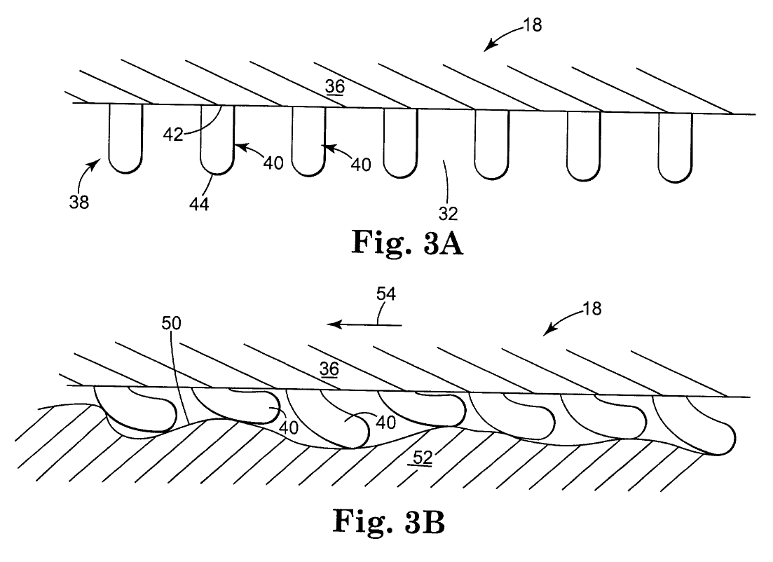

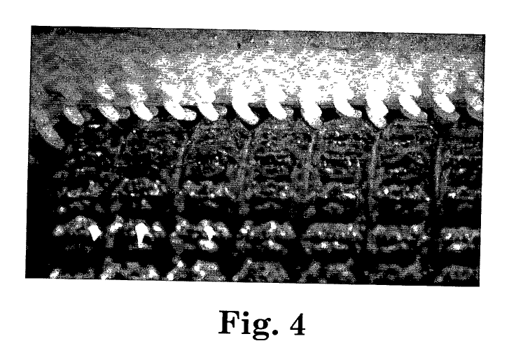

FIG. 3A illustrates the first side 32 of the layer of frictional material 18, showing the array of upstanding stems 38 thereon. Each stem 40 has a fixed end 42 (which is affixed to or integral with the base layer 36) and a free end 44. FIG. 3B illustrates the reactions of the stems 38 when urged against a surface 50 of an article 52 under a normal force, and further under the application of a shear force (in direction of arrow 54). As seen in FIG. 3B, the stems 40 bend to accommodate irregularities in the surface 50, with side walls of the stems 40 engaging the surface 50 (along with the free end 44 of each stem 40) to increase the engaged surface area between the layer of frictional material 18 and article 52. This increased surface area is achieved in either wet or dry conditions, and each stem acts independently (as illustrated in FIG. 3B and also in the enlarged photograph of FIG. 4) to achieve the highest possible surface area contact between the opposed materials. This is important in view of the fact that the contact between the finger-borne layer of frictional material 18 is generally non-planar (i.e., following irregular finger surface contours) when engaged with a likewise non-uniform surface such as a tool handle or surface of a ball, so that under shear, the relative surface area contact between the two opposed surfaces at their frictional interface is maximized.

A suitable frictional material for this purpose includes the gReptile™ brand material available from 3M Company, St. Paul, Minn., identifiable as product number G400. This material is more specifically described U.S. Pat. No. 6,372,323, and in Applicant's co-pending U.S. patent application Ser. No. 09/637,567, both of which incorporated by reference herein, and is specifically described in the examples below for a fingertip gripping pad application.

The embodiment of the fingertip gripping pad 10 shown in FIGS. 1 and 2 is shaped to accommodate a user's finger and to minimize bunching and wrinkling of the materials forming the pad 10. Those materials and the pad itself must of course be somewhat flexible to accommodate the bending of the user's finger. As seen in FIG. 1, the backing sheet has a central section 60 (which corresponds generally to the friction zone 30). A plurality of opposed tabs extend outwardly from the central section 60. The embodiment of FIG. 1 has three pairs of opposed tabs, tabs 62A and 62B, tabs 64A and 64B, and tabs 66A and 66B. A generally slotted opening or gap between adjacent tabs is formed to facilitate finger movement once the fingertip gripping pad 10 is in place on a finger.

Each pair of opposed tabs is long enough to extend at least half way around the user's finger. In one embodiment, the opposed tabs are long enough to extend completely around the user's finger, with a slight overlap at the free ends of the tabs. As seen in FIG. 1, the central section 60 of the backing sheet 12 is elongated in direction generally normal to the opposed pairs of tabs. The central section 60, as defined by the friction zone 30, has, along its elongated direction, an intermediate segment 68 of reduced width. At the intermediate segment 68, the layer of frictional material 18 is shaped to be necked inwardly, adjacent an area where the tabs are separated by gaps, to accommodate placement of the fingertip gripping pad 10 along the knuckles of a finger and motion of the finger once in place. The backing sheet 12 and layer of frictional material 18 both have, in the embodiment illustrated in FIG. 1, rounded edges, and those components are shaped so that their defined friction zone 30 covers the primary contact pads on the user's finger.

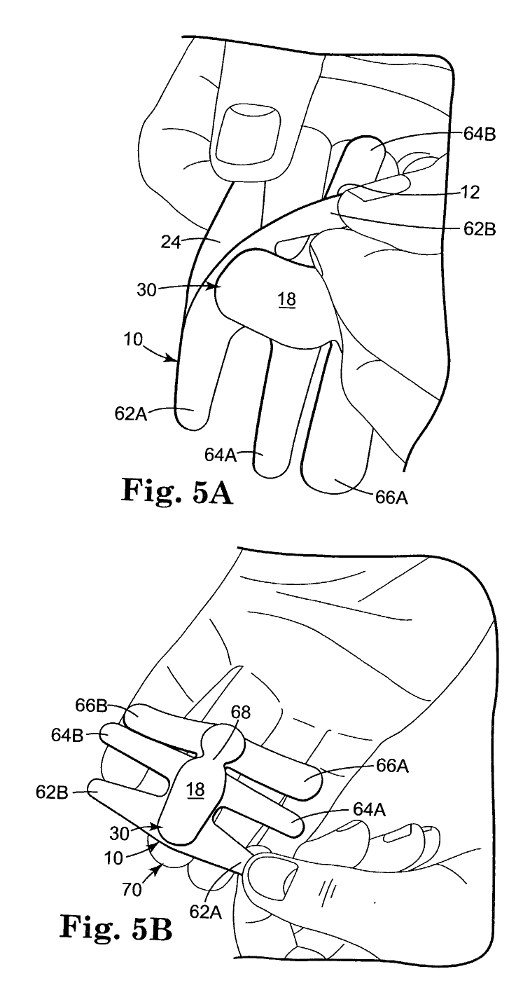

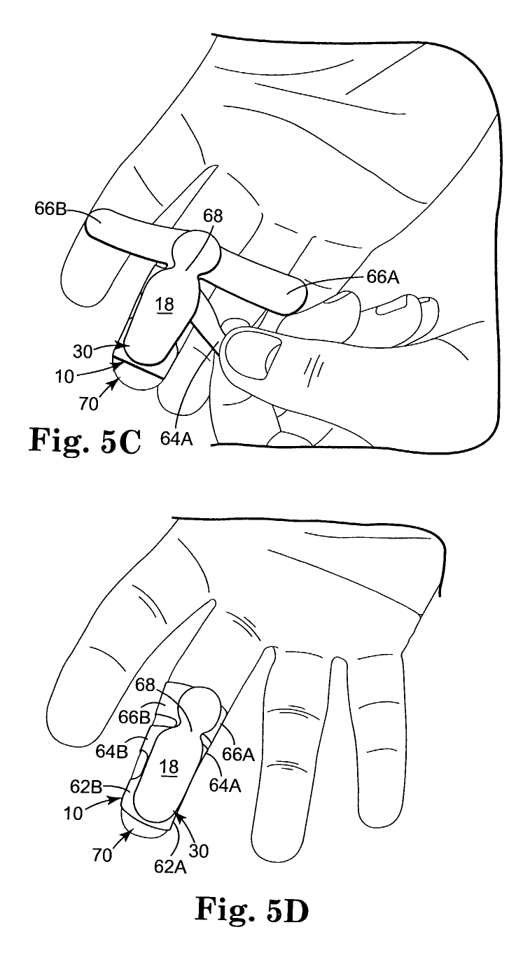

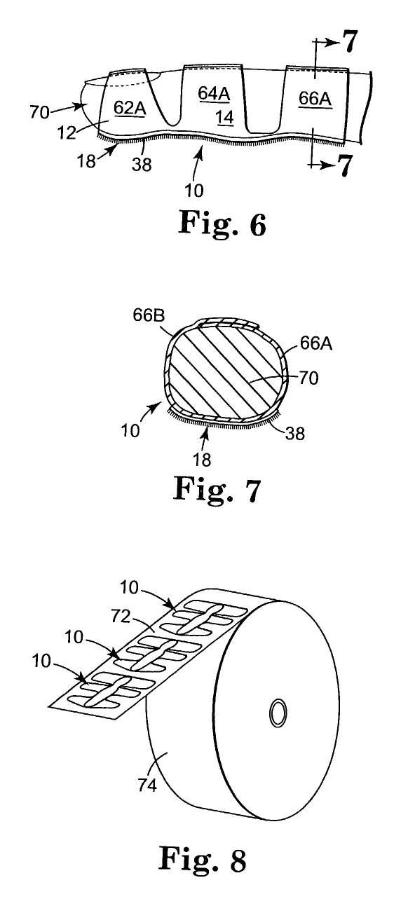

FIGS. 5A to 5D illustrate the application of a fingertip gripping pad 10 to a user's finger 70. In FIG. 5A, the adhesive liner 24 is first removed from the second side 16 of the backing sheet 12 and discarded, thus exposing the adhesive 22. The fingertip gripping pad 10 is then aligned relative to the user's finger 70 to place the layer of frictional material 18 in a desired position for use on the finger 70, as seen in FIG. 5B. As referenced above, the fingertip gripping pad 10 is aligned so that the opposed tab pairs do not extend over knuckles of the finger, and the layer of frictional material 18 is aligned to accommodate finger movement, and to expose desired portions of the finger, i.e., the fingertip. Prior to application of the fingertip gripping pad 10 to the user's finger 70, the finger may be cleaned or wiped to remove excess oils or other fluids thereon, in order to increase adhesion. Each tab of the backing sheet 12 is then wrapped about the user's finger 70. FIG. 5C illustrates this process, showing tabs 62A, 62B and 64B already wrapped and adhered to the user's finger, with the user in the process of wrapping tab 64A about the finger (and tabs 66A and 66B have not yet been adhered to the user's finger 70). FIG. 5D shows the fingertip gripping pad 10 completely secured to a user's finger 70, as does FIG. 6. FIG. 7 illustrates the overlap of free ends of the tabs 66A and 66B in this embodiment. As the tabs are wrapped about the user's finger 70, the user presses down on the first side 14 of each tab of the backing sheet 12, thereby urging the pressure sensitive adhesive 22 into adhering contact with the finger 70. The fingertip gripping pad 10 is thus mounted and ready for use. Once the task has been completed, the pad 10 is removed from the user's finger 70, in the same manner as an adhesive bandage might be removed (i.e., peeled off and disposed of).

Each fingertip gripping pad 10, as seen in FIGS. 1, 2 and 5-7, can be mounted on its own liner, which has the same general shape as the backing sheet 12. Alternatively, a plurality of fingertip gripping pads 10 can be mounted on a common liner. FIG. 8 illustrates one form of dispensing fingertip gripping pads 10 from a common liner 72 which may be wound into roll form 74.

In use, a user may place a fingertip gripping pad 10 on only one finger, or two fingers, or on several fingers, on one or both of the user's hands. Each inventive fingertip gripping pad conforms to its respective finger's surface and presents a friction zone 30 of a desired shape, which itself is designed to conform to the surface of an article being gripped, and provide a high coefficient of friction, in either wet or dry conditions. If the article being gripped (such as a tool handle, railing or racquet handle) also has a layer of frictional material thereon, even greater frictional interface is possible between the two materials, as the opposed arrays of upstanding stems interengage to define an even greater and more cooperative surface area for frictional interface.

In one embodiment, the desired coefficient of friction for the friction zone 30 (first side 32 of layer of frictional material 18) is at least 1.1. It is fundamental in operation that the gripping pad 10 not become dislodged in use, and with such a high coefficient of friction, the friction zone 30 presented is relatively “sticky” or “tacky.” Accordingly, it is important that the gripping pad 10 be firmly affixed to the user's finger. A load ratio evaluation was conducted to define an acceptable relationship between the shear force characteristic of the first side 32 of the layer of frictional material 18 and the shear force characteristic of adhesion between the second side 16 of the backing sheet and the user's finger. In order to firmly affix the gripping pad 10 to a user's finger in view of the high coefficient of friction forces encountered in use, a design load to anticipated load ratio of 1.3:1 or higher is desired, in order to assure durability and continued engagement of the fingertip gripping pad 10 in use. The tests used to determine the coefficient of friction of the frictional material 18 and load values for the respective materials are outlined in the examples below, for the specific materials evaluated.

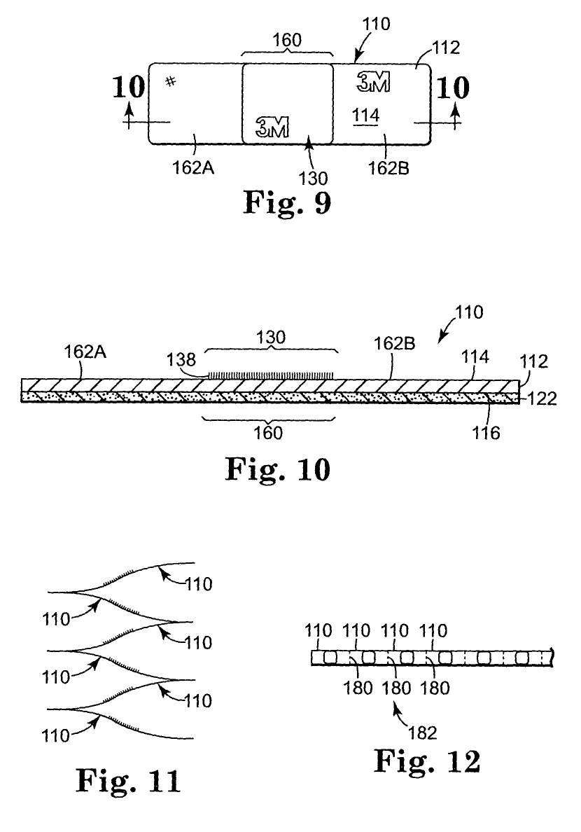

FIGS. 9 and 10 illustrate another embodiment of the fingertip gripping pad of the present invention, referenced as gripping pad 110. In this embodiment, backing sheet 112 has a central section 160 and one pair of opposed tabs 162A and 162B. A friction zone 130 is again defined adjacent to the central 160 of the backing sheet 112, on a first side 114 thereof. As illustrated in FIG. 10, there is no separate layer of frictional material in this embodiment. The frictional material is instead defined as friction zone 130 which is formed integrally with the backing sheet 112. In other words, when the friction zone 130 is defined by an array of upstanding stems 138 like disclosed above, the stems are integral with the backing sheet 112 and project therefrom. Materials useful for this purpose are the same as those disclosed above with respect to the embodiment of FIGS. 1-7.

Alternatively, the array of stems 138 in this embodiment could be formed separately from the base sheet 112 and affixed thereto, such as described in the context of the embodiment of FIGS. 1-7 disclosed above.

On a second side 116 of the backing sheet 112, pressure sensitive adhesive 122 is provided. As discussed above, with respect to the embodiment of FIGS. 1-2, the tabs 162A and 162B project outwardly from the central section 160, and are sufficiently long to extend (together) at least half way around the user's finger. In one embodiment, the tabs are long enough to extend completely around a user's finger, and even slightly overlap adjacent the free ends thereof.

In this embodiment, no adhesive liner is provided for the adhesive 122. The fingertip gripping pads 110 can thus be dispensed in a Z-fold arrangement, as illustrated in FIG. 11. To do so, the adhesive 122 is either made more aggressive at alternate ends of adjacent pads 110 in a stack, or portions of the first side 114 and friction zone 130 are provided with an adhesive release layer coating, in order to obtain a differential adhesion between adjacent pads 110 in a stack of pads 110. The fingertip gripping pads 110 can then be dispensed in a Z-fold style, similar to that illustrated for the article sheets shown in U.S. Pat. Nos. 4,907,825 and 4,416,392. Alternatively, as illustrated in FIG. 12, the fingertip gripping pads 110 can be dispensed in strip or roll form, with the pads 110 connected end-to-end longitudinally, and then separated for use. In one embodiment, such separation is facilitated by perforations 180 between adjacent pads 110 in a strip 182. In yet another embodiment, the fingertip gripping pads may be formed in strip or roll form with each pad extending laterally across the strip or roll of material. For use, an individual pad is then separated from a free end of the strip or roll and applied.

Numerous modifications can be made to the present invention while still achieving the desired result. For example, the friction zone can have any desired shape, depending upon the particular use and/or appendage to which the inventive apparatus will be applied. In addition, the coefficient of friction in the friction zone may be increased for certain applications, or may be varied in certain areas on the same apparatus (e.g., the coefficient of friction may be made higher adjacently a distal fingertip portion of the friction zone while lower on the same apparatus in a more proximal area of the friction zone). While the illustrated embodiments disclose the use of a pressure sensitive adhesive for adhering the backing sheet to a user's appendage, other means may be employed for that purpose. For example, a two part mechanical fastener may be employed for that purpose. One portion of the mechanical fastener may be adhered to the second side of the backing sheet, while the other (mating) portion of the mechanical fastener may be otherwise secured to a user's appendage (e.g., directly to the user's skin), or to an article of clothing (e.g., a vest, glove, boot, etc.). In yet another embodiment, the backing sheet itself may be defined as an elastic band sized to stretchably extend around the user's appendage (e.g., an arm band or a wrist band) with the friction zone defined on a first outer side thereof. Other modifications and combinations of the various features disclosed herein would be apparent to one of ordinary skill in the art.

In order to evaluate and further define the features of the present invention, examples were conducted and tested as follows:

Test Method 1—Static Coefficient of Friction. This test method was employed to determine the coefficient of starting friction of the frictional material. The procedure was conducted in accordance with ASTM test procedure D1894-01, using a moving sled and a stationary plane as described in system configuration ‘C’ with a moving gage. The stationary plane was a machined cold rolled steel plate with smooth anodized surface texture. The apparent contact pressure exerted on the test specimen by the sled was one lb. (0.45 kg). The x-head speed of the gage was 5 cm/s. The test specimen was conditioned at a room temperature of 22° C. and relative humidity of 65%.

Test Method 2—Strength Properties of Adhesives in Shear. This test method was employed to determine the shear strength of an adhesive in accordance with ASTM test procedure D3165-00. The metal used in forming the test joint was cold rolled steel and was prepared by cleaning with isopropyl alcohol. The adhesive was applied by transfer pressure sensitive adhesive film and resulted in a contact area of 1.61 cm² and length of overlap of 1.27 cm. Testing was performed at a temperature of 22° C., and relative humidity of 65%. Loading rate of the specimen was 25.4 mm/min. Failure load is expressed in kPa of shear area.

### EXAMPLE BACKGROUND

Examples of the invention configured as fingertip gripping pads were tested, similar to those illustrated in FIGS. 1 and 2. With these examples, the act of gripping involves loading a grip surface and drawing it over a contact surface. In the below example of the invention, a grip, with outwardly exposed grip surface, is removably attached to a substrate such as skin. Drawing a grip surface of the invention against a contact surface results in desirably high shear force between the grip and the contact surface. The shear force generated between the grip and contact surface translates to the attachment interface as a counter force. If the shear strength between the attachment surface and substrate is less than the counter force, the grip will be stripped from the substrate. For a given activity, one that would employ a grip element of the invention, a careful balance between the anticipated gripping shear and shear strength of the attachment must be achieved. Attention to this limiting balance is particularly acute when the grip element is attached to skin, where its effective attachment is impacted by substrate movement, moisture, repeated shear incidents, and the irregular nature of the attachment surface, and are limited by an acceptable ultimate level of adhesion.

Forces held in balance in the design of the example of the invention are described by the characteristics of the materials employed and configuration of the grip. Considering a grip with a grip surface area, A_{g} encountering a gripping shear S_{g}, with an attachment surface Aₐ, and attachment shear strength Sₐ, the following design criterion should be met:

SₐAₐ≧S_{g}A_{g} Equation 1

For a given loading L on the gripping surface, the maximum gripper shear stress S_{g} that could be reached can be related to the static coefficient of friction μₛ of the gripping material by:

S_{g}=μₛL Equation 2

Combining Equations 1 and 2 gives the relationship between the shear properties of the grip and attachment surfaces as they relate to the configuration of the grip element, and can be expressed by:

L≦(Aₐ/A_{g})(Sₐ/μₛ) Equation 3

In examples of the invention the design load L_{d}, given by Equation 3 would preferably be four times the anticipated maximum load. The anticipated maximum load of the grip, in its intended use environment, would be predicted by estimating the maximum load the grip element would encounter. As an example, the possible load that could be exerted by a person's fingertip might range between 50 and 500 kPa. This load would represent the anticipated load range that would be experienced on a grip element intended to cover the fingertip of a user. It should be noted that the shear strength of the attachment might result from adhesive or mechanical attachments that can be readily applied and removed. The ratio of the design load to the anticipated load must always be greater than 1:1, or the adhesion of the gripping pad to the user's finger will fail in use. In examples of the invention, the design load of the grip will be a 4×factor over the anticipated maximum load.

The following example illustrates the relationship between the design and anticipated load factors for a fingertip grip of the present invention in which the grip element is designed to be adhesively attached to the end of a user's finger.

### Example 1

A gripping apparatus of the invention was designed for application as a fingertip grip-enhancing element. The element was constructed using a gripping surface that was bonded to a flexible bandage backing coated with a pressure sensitive adhesive. The gripping surface (i.e., frictional material) was cut from a sheet of gReptile™ gripping material type G400, available from 3M Company, St. Paul, Minn. This frictional material had a gripping surface topography on one face of small protruding flexible pins arranged in an ordered pattern with 465 pins per cm² and had a static coefficient of friction of 1.12, as determined by Test Method 1. To form the desired friction zone, the frictional material was cut into a generally oval shape with a rule die giving a grip surface area of 6.5 cm². The smooth face of the frictional material was adhesively affixed to a top surface of the backing using a high strength adhesive, Scotch® type 300LSE, available from 3M Company, St. Paul, Minn. The cut frictional material was positioned on the top surface of the backing as is shown in FIG. 1. The material used as the attachment component (i.e., the backing sheet), was a bandage tape, Comfort Tape, type 43910008383, available from 3M Company, St. Paul, Minn. The bandage tape had a pressure sensitive adhesive covering one face and was cut into the configuration illustrated in FIG. 1, which resulted in an adhesive attachment surface area of 24.3 cm². The shear strength of the adhesive was determined to be 116.4 kPa by Test Method 2.

In use, the gripping element of the example might be employed as a finger gripping aid in a sport activity such as basketball where the anticipated contact force might be between 50 and 300 kPa. A summary of the anticipated and design contact force; gripping and attachment contact areas; and shear strength and coefficient of friction properties of the materials used are summarized in Table 1, where:

A_{g}=grip surface area (i.e., friction zone area);

Aₐ=attachment shear area (i.e., area of adhesive on the second side of the backing sheet);

μₛ=static coefficient of friction (of the frictional material);

Sₐ=attachment shear strength (of the adhesive);

L_{d}=design load; and

Lₐ=anticipated load.

TABLE 1

| Contact Areas | | Shear | | Load Limits | | Load |
|---|---|---|---|---|---|---|
| (cm²) | | Characteristics | | (kPa) | | Ratio |
| A_{g} | Aₐ | μₛ | Sₐ (kPa) | L_{d} | Lₐ | L_{d}:Lₐ |
| 6.5 | 24.3 | 1.12 | 116 | 388 | 50 | 7.8:1 |
| 6.5 | 24.3 | 1.12 | 116 | 388 | 300 | 1.3:1 |

As is indicated in Table 1, a load ratio of 1.3:1 to 7.8:1 would be required to prevent stripping of the grip element of Example 1 from the finger during use. If a mechanical fastener type of attachment was used to attach the gripping surface might be expected to be in the range of 4:1. Regardless of the application, the load ratio L_{d}:Lₐ would always be at least greater than 1:1.

Although the present invention has been described with reference to preferred embodiments, workers skilled in the art will recognize that changes may be made in form and detail without departing from the spirit and scope of the invention.

1. An apparatus for presenting an area of desired frictional interface on a portion of a user's appendage which comprises:
a flexible layer of material having first and second opposite sides, with the first side having a frictional engagement surface;
a flexible backing sheet having first and second opposite faces, the backing sheet having a central section and opposed tab portions, with the first face of the backing sheet affixed to the second side of the flexible layer of material, in at least a portion of the central section of the backing sheet; and
a pressure sensitive adhesive disposed on the second face of the backing sheet, wherein the tab portions of the backing sheet are sufficiently long, in dimension extending away from the central section, so that when the second face of the backing sheet is adhered to a user's appendage, the tab portions overlap at free ends thereof.

2. The apparatus of claim 1 wherein the pressure sensitive adhesive is disposed over the entire second face of the backing sheet.

3. The apparatus of claim 1 wherein the backing sheet has a plurality of pairs of opposed tab portions.

4. The apparatus of claim 3 wherein the pairs of tab portions extend generally parallel to one another.

5. The apparatus of claim 1 wherein each tab has a generally constant lateral dimension along its length.

6. The apparatus of claim 1 wherein the friction engagement surface is defined by an array of flexible polymeric stems.

7. The apparatus of claim 6 wherein each stem is a generally cylindrical post.

8. The apparatus of claim 1 wherein the frictional engagement surface has a coefficient of friction of at least 1.1.

9. The apparatus of claim 1 wherein the central section of the backing sheet is elongated in direction generally normal to the opposed tab portions.

10. The apparatus of claim 9 wherein the central section has, along its elongated direction, an intermediate segment of reduced width.

11. The apparatus of claim 1 wherein the backing sheet is elastic.

12. The apparatus of claim 1, and further comprising:
a removeable liner sheet for masking the adhesive prior to application of the apparatus to a user's appendage.

13. The apparatus of claim 12 wherein the liner sheet has the same shape as the backing sheet.

14. The apparatus of claim 12 wherein the liner sheet has a plurality of backing sheets adhered thereto.

15. The apparatus of claim 14 wherein the liner sheet is wound in a roll.

16. The apparatus of claim 6 wherein each stem extends generally perpendicularly from the backing sheet when applied on a user's appendage.

17. The apparatus of claim 6 wherein, under an applied shear force, the stems deflect to increase the exposed surface area thereof for enhanced frictional interface.

18. The apparatus of claim 1 wherein the tab portions have indicia thereon, on the first faces thereof.

19. The apparatus of claim 1 wherein the layer of material has indicia thereon, on the first side thereof.

20. The apparatus of claim 1 wherein the frictional engagement surface is operative to define a desired frictional interface when wet or dry.

21. An apparatus for presenting an area of desired frictional interface on a portion of a user's appendage which comprises:
a flexible layer of material having first and second opposite sides, with the first side having a frictional engagement surface;
a flexible backing sheet having first and second opposite faces, the backing sheet having a central section and opposed tab portions, with the first face of the backing sheet affixed to the second side of the flexible layer of material, in the central section of the backing sheet; and
wherein the tab portions of the backing sheet are sufficiently long, in dimension extending away from the central section, so that when the second face of the backing sheet is adhered to a user's appendage, the tab portions extend at least half way around the appendage.

22. An apparatus for disposing a desired frictional interface on a user's appendage which comprises:
a flexible backing sheet having first and second opposite faces, the backing sheet having a central section and opposed tab portions;
a pressure sensitive adhesive disposed on the second face of the backing sheet wherein the tab portions of the backing sheet are sufficiently long, in dimension extending away from the central section, so that when the second face is adhered to a user's appendage, the tab portions extend at least half way around the appendage; and
a friction zone defined on the first face of the backing sheet, the zone having a static coefficient of friction of at least 1.1.

23. An apparatus for disposing a desired frictional interface on a portion of a user's appendage which comprises:
a flexible backing sheet having first and second opposite faces;
means for removeably attaching the second face of the flexible backing sheet to a user's appendage; and
a friction zone defined on the first face of the backing sheet, the friction zone having a static coefficient of friction of at least 1.1,
wherein the apparatus has a load ratio between a shear force design load and an anticipated load of at least 1.3:1.

24. The apparatus of claim 23 wherein the removeably attaching means is selected from one of the group of a pressure sensitive adhesive on the second face of the backing sheet, an opposed two part mechanical fastener with one part thereof disposed on the second face of the backing sheet and the other part thereof disposed on the user's appendage, and the backing sheet comprising an elastic band sized to stretchably extend around the user's appendage.

25. A method for presenting a desired frictional interface on a user's appendage, comprising the steps of:
providing a flexible backing sheet having first and second opposite faces, the backing sheet having a central section and a plurality of tab portions extending outwardly from the central section, the central section having, on its first face, a friction zone having a static coefficient of friction of at least 1.1;
exposing the pressure sensitive adhesive on the second face of the backing sheet;
aligning the backing sheet relative to a user's appendage for application; and
pressing the exposed pressure sensitive adhesive against the user's appendage to adhere the backing sheet thereto.

26. The method of claim 24 wherein the aligning step includes extending the tab portions at least half way around the user's appendage.

27. The method of claim 24 wherein the aligning step includes extending two of the tab portions around the user's appendage so that free ends of those two tab portions overlap.

28 A method for presenting a desired frictional interface between a user's appendage and an article, comprising the steps of:
securing a bottom face of a flexible backing sheet to a user's appendage, wherein the backing sheet has, on a top face opposite the bottom face, a friction zone having a static coefficient of friction of at least 1.1; and
contacting an article with the friction zone for manipulation of the article.

29. The method of claim 28 wherein the securing step includes adhering the bottom face of the flexible backing sheet to a user's appendage.

30. The method of claim 28 wherein the backing sheet has a central section and a tab portion extending outwardly from the central section, wherein the friction zone is on the central section.

31. The method of claim 30 wherein the backing sheet has at least two tab portions extending outwardly from the central section.
